(19) United States
(12) Patent Application Publication
(10) Pub. No.: US 2005/0244839 A1
(43) Pub. Date: Nov. 3, 2005

(54) Granulin-epithelin precursor (GEP) overexpression as a target for diagnosis, prognosis and treatment of hepatocellular carcinoma (HCC)

(76) Inventors: Siu Tim Cheung, Apleichau (HK); Sheung Tat Fan, Hong Kong (HK)

Correspondence Address: Robert D. Katz;Cooper & Dunham LLP, 1185 Avenue of the Americas, New York, NY, 10036, US

(21) Appl. No.: 10/836,390

(22) Filed: Apr. 29, 2004

(51) Int. Cl. C12Q 1/68; G01N 33/574

(52) U.S. Cl. 435/6; 435/7.23

(57) ABSTRACT

This invention further provides methods for determining whether an agent causes a reduction in the activity of a Granulin-Epithelin Precursor (GEP) protein in a cell. This invention also provides methods for reducing the expression of Granulin-Epithelin Precursor (GEP) protein in a cell. This invention also provides methods for determining whether a subject is afflicted with Hepatocellular carcinoma (HCC). This invention provides methods for determining whether a subject is afflicted with Hepatocellular carcinoma (HCC) This invention further provides a method for treating a subject afflicted with Hepatocellular carcinoma (HCC) comprising administering to the subject a therapeutically effective amount of an agent which specifically interferes with the expression of the Granulin-Epithelin Precursor (GEP) protein in the tumor cells of the subject.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND OF THE INVENTION

Hepatocellular carcinoma (HCC) is the fifth most common cancer worldwide, with about half a million new cases and almost as many deaths per year.¹⁻³ Better understanding of the etiological factors and molecular basis of the disease is crucial in disease prevention and management. Epidemiological studies have shown that hepatitis B and C virus infections, alcohol-induced liver injury and consumption of aflatoxin are closely associated with HCC. However, little is known about the molecular basis of liver cancer development and progression. The p53 tumor suppressor gene is believed to play a major role as ‘the cellular gatekeeper’ while β-catenin oncogene deregulation has recently demonstrated neoplastic transformation potential.^{2,4} However, the major growth factor in liver carcinogenesis is largely unknown.

Differentially expressed genes between HCC and liver tissue adjacent to HCC have recently been identified.⁵ Granulin-epithelin precursor (GEP) is one of the highly expressed genes in HCC with gene locus at 17q21.32. GEP protein is a secretory protein capable of stimulating cell proliferation, and its reduced expression is associated with inhibition of tumorigenic potential.^{7,8} Chromosome gain at 17q is detected in 30-60% of liver cancers,^{9,10} strongly suggesting the presence of growth factor(s)/proto-oncogene(s) at this chromosome arm. No studies have reported GEP expression pattern and its biological role in HCC. In this study, the RNA level and protein localization of GEP in HCCs, liver tissues adjacent to the HCCs, and normal liver tissues were examined.

### SUMMARY OF THE INVENTION

This invention provides a method for determining whether an agent causes a reduction in the expression of Granulin-Epithelin Precursor (GEP) protein in a cell, comprising the steps of (a) contacting the cell with the agent under conditions which, in the absence of the agent, permit expression of the GEP protein; (b) after a suitable period of time, determining the amount of expression in the cell of the GEP protein; and (c) comparing the amount of expression determined in step (b) with the amount of expression which occurs in the absence of the agent, whereby a reduced amount of expression in the presence of the agent indicates that the agent causes a reduction in the expression of the GEP protein.

This invention further provides a method for determining whether an agent causes a reduction in the activity of a Granulin-Epithelin Precursor (GEP) protein in a cell, comprising the steps of (a) contacting the cell with the agent under conditions which, in the absence of the agent, permit activity of the GEP protein; (b) determining the amount of activity of the GEP protein in the cell; and (c) comparing the amount of activity determined in step (b) with the amount of activity which occurs in the absence of the agent, whereby a reduced amount of activity in the presence of the agent indicates that the agent causes a reduction in the activity of the GEP protein.

This invention also provides a method for reducing the expression of Granulin-Epithelin Precursor (GEP) protein in a cell comprising introducing into the cell an agent which specifically interferes with the expression of the GEP protein in the cell.

This invention also provides a method for determining whether a subject is afflicted with Hepatocellular carcinoma (HCC) comprising the steps of (a) determining the level of Granulin-Epithelin Precursor (GEP) protein expression in the tumor cells of the subject; (b) determining the level of Granulin-Epithelin Precursor (GEP) protein expression in the normal liver cells of the subject; and (c) comparing the level of expression determined in step (a) with the level of expression determined in step (b), wherein a higher expression level in step (a) indicates that the subject is afflicted with HCC.

This invention also provides a method for determining whether a subject is afflicted with Hepatocellular carcinoma (HCC) comprising the steps of (a) determining the level of Granulin-Epithelin Precursor (GEP) protein expression in the tumor cells of the subject; and (b) comparing the level of expression determined in step (a) with the level of expression of GEP protein in normal liver cells of a healthy subject, wherein a higher expression level in step (a) indicates that the subject is afflicted with HCC.

This invention also provides a method for determining whether a subject is afflicted with Hepatocellular carcinoma (HCC) comprising the steps of: (a) determining the amount of Granulin-Epithelin Precursor (GEP)-encoding mRNA in the tumor cells of the subject; (b) determining the amount of Granulin-Epithelin Precursor (GEP)-encoding mRNA in the normal liver cells of the subject; and (c) comparing the amount of mRNA determined in step (a) with the amount of mRNA determined in step (b), wherein a greater amount of mRNA in step (a) indicates that the subject is afflicted with HCC.

This invention also provides a method for determining whether a subject is afflicted with Hepatocellular carcinoma (HCC) comprising the steps of (a) determining the amount of Granulin-Epithelin Precursor (GEP)-encoding mRNA in the tumor cells of the subject; and (b) comparing the amount of mRNA determined in step (a) with the amount of GEP-encoding mRNA found in normal liver cells of a healthy subject, wherein a greater amount of mRNA in step (a) indicates that the subject is afflicted with HCC.

This invention further provides a method for treating a subject afflicted with Hepatocellular carcinoma (HCC) comprising administering to the subject a therapeutically effective amount of an agent which specifically interferes with the expression of the Granulin-Epithelin Precursor (GEP) protein in the tumor cells of the subject.

### BRIEF DESCRIPTION OF THE FIGURES

FIG. 1A-D GEP expression in human liver samples. (A) RNA quantization by real-time RT-PCR. The top and bottom horizontal lines of the box indicate the 25^{th} and 75^{th} percentiles, respectively. The lines within the box indicate the median values. The top and bottom horizontal bars indicate data within 1.5 times the interquartile range. (B-D) Immunohistochemical staining of GEP. HCC with protein signal score 3 (B), liver adjacent to HCC with protein score 0 (C), and normal liver tissue with protein score 0 (D).

FIG. 2A-D GEP and p53 protein localization in HCC. (A) GEP protein staining. Tumor regions with strong GEP expression were indicated by arrows (×40 magnification). (B) p53 protein staining. Tumor regions with p53 nuclei expression were indicated by arrow heads (×40). (C) GEP protein staining for the enlarged magnification of the boxed area (×200). (D) p53 protein staining for the enlarged magnification of the boxed area (×200). Protein signals were stained in brown, and the sections were counter-stained with hematoxylin.

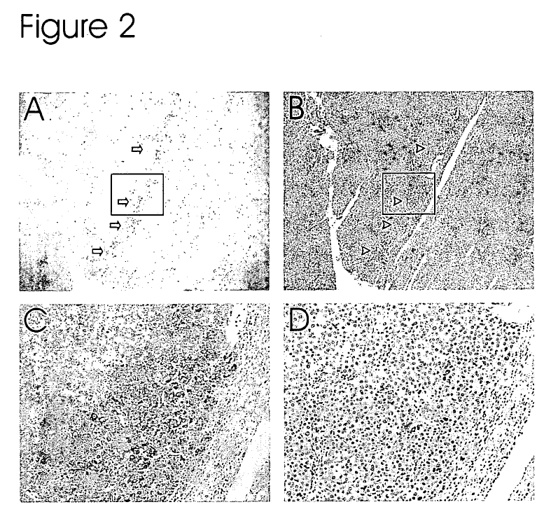

FIG. 3A-C A reduced GEP level decreased the cell proliferation rate and cell activity.

Transfectants of Hep3B and Huh7 cells were examined in serum-containing or serum-limited condition: Δ vector control (V), ▴ anti-sense (AS), ▪ full-length (FL), □ sense control (S), and ♦ parental cell line. (A) GEP protein levels. (B) Cell growth curves. (C) Cell activity by MTT assays.

FIG. 4A-C A reduced GEP level decreased the tumor invasion ability, colony-forming ability and tumorigenic potential. (A) Invasion ability of the cells was examined by the Matrigel invasion chamber. (B) Colony formation ability on soft agar. (C) Tumorigenic potential in athymic nude mice.

### DETAILED DESCRIPTION OF THE INVENTION

Definitions

As used in this application, except as otherwise expressly provided herein, each of the following terms shall have the meaning set forth below.

As used herein, “administering” an agent can be effected or performed using any of the various methods and delivery systems known to those skilled in the art. The administering can be performed, for example, intravenously, via cerebrospinal fluid, orally, nasally, via implant, transmucosally, transdermally, intramuscularly, and subcutaneously.

As used herein, “agent” shall mean any chemical entity, including, without limitation, a protein, an antibody, a nucleic acid, a small molecule, and any combination thereof.

As used herein, “antibody” shall include, by way of example, both naturally occurring and non-naturally occurring antibodies. Specifically, this term includes polyclonal and monoclonal antibodies, and antigen-binding fragments thereof. Furthermore, this term includes chimeric antibodies (e.g., humanized antibodies) and wholly synthetic antibodies, and antigen-binding fragments thereof.

As used herein, “antisense molecule” shall mean any nucleic acid which, when introduced into a cell (directly or via expression of another nucleic acid directly introduced into the cell), specifically hybridizes to at least a portion of an mRNA in the cell encoding a protein (i.e., target protein) whose expression is to be inhibited, and thereby inhibits the target protein's expression.

As used herein, “DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain.

As used herein, “pharmaceutically acceptable carrier” shall mean any of the various carriers known to those skilled in the art.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

As used herein, “small interfering RNA” (also referred to as siRNA or RNAi) includes, without limitation, a polynucleotide sequence identical or homologous to a target gene (or fragment thereof) linked directly, or indirectly, to a polynucleotide sequence complementary to the sequence of the target gene (or fragment thereof). The siRNA optionally comprises a polynucleotide linker sequence of sufficient length to allow for the two polynucleotide sequences to fold over and hybridize to each other. The linker sequence is designed to separate the antisense and sense strands of siRNA significantly enough to limit the effects of steric hindrances and allow for the formation of a dsRNA molecule, and not to hybridize with sequences within the hybridizing portions of the dsRNA molecule. siRNA is discussed, e.g., in U.S. Pat. No. 6,544,783).

As used herein, “subject” shall mean any animal, such as a human, non-human primate, mouse, rat, guinea pig or rabbit.

As used herein, “suitable period of time” shall mean, with respect to the instant methods, an amount of time sufficient to permit the effect of the agent.

As used herein, “therapeutically effective amount” means an amount sufficient to treat a subject afflicted with Hepatocellular carcinoma (HCC).

As used herein, “treating” Hepatocellular carcinoma (HCC) shall mean slowing, stopping or reversing the disease progression.

### EMBODIMENTS OF THE INVENTION

This invention provides a method for determining whether an agent causes a reduction in the expression of Granulin-Epithelin Precursor (GEP) protein in a cell, comprising the steps of (a) contacting the cell with the agent under conditions which, in the absence of the agent, permit expression of the GEP protein; (b) after a suitable period of time, determining the amount of expression in the cell of the GEP protein; and (c) comparing the amount of expression determined in step (b) with the amount of expression which occurs in the absence of the agent, whereby a reduced amount of expression in the presence of the agent indicates that the agent causes a reduction in the expression of the GED protein. In one embodiment, the cell is present in a cell culture. In another embodiment, the cell is a tumor cell.

In a further embodiment, determining the amount of expression is performed by determining the amount of GEP protein-encoding mRNA in the cell. In another embodiment, determining the amount of expression is performed by determining the amount of GEP protein in the cell. The determining of the amount of GEP protein in the cell may be performed using an antibody specific for the GEP protein.

This invention further provides a method for determining whether an agent causes a reduction in the activity of a Granulin-Epithelin Precursor (GEP) protein in a cell, comprising the steps of (a) contacting the cell with the agent under conditions which, in the absence of the agent, permit activity of the GEP protein; (b) determining the amount of activity of the GEP protein in the cell; and (c) comparing the amount of activity determined in step (b) with the amount of activity which occurs in the absence of the agent, whereby a reduced amount of activity in the presence of the agent indicates that the agent causes a reduction in the activity of the GEP protein. In one embodiment, the cell is present in a cell culture. In another embodiment, the cell is a tumor cell.

This invention also provides a method for reducing the expression of Granulin-Epithelin Precursor (GEP) protein in a cell comprising introducing into the cell an agent which specifically interferes with the expression of the GEP protein in the cell. In one embodiment, the cell is present in a cell culture or is a tumor cell. In another embodiment, the agent is a nucleic acid. The nucleic acid may be, but is not limited to, a small interfering RNA, a ribozyme, a DNAzyme or an antisense molecule. The antisense molecule may comprises the nucleic acid sequence GAAGGGGCAG CAACTGGAAG TCCCTGAGAC GGTAAAGATG CAGGAGTGGC CGGCAGAGCA GTGGGCATCA ACCTGGCAGG GGCCACCCAG ATGCCTGCTC AGTGTTGTGG GCCATTTGTC CAGAAGGGGA CGGCAGCAGC TGTAGCTGGC TCCTCCGGGG TCCAGGCAGC AGGCCACAGG GCAGAACTGA CCATCTGGGC ACCGCGTTCC AGCCACCAGC CCTGCTGTTA AGGCCACCCA GCTCACCAGG GTCCACATGG TCTGCCTGCG TCCGACTCCG CGGTCCTTG as set forth in SEQ ID NO:5.

This invention also provides a method for determining whether a subject is afflicted with Hepatocellular carcinoma (HCC) comprising the steps of (a) determining the level of Granulin-Epithelin Precursor (GEP) protein expression in the tumor cells of the subject; (b) determining the level of Granulin-Epithelin Precursor (GEP) protein expression in the normal liver cells of the subject; and (c) comparing the level of expression determined in step (a) with the level of expression determined in step (b), wherein a higher expression level in step (a) indicates that the subject is afflicted with HCC. In one embodiment, the level of expression of GEP protein is determined by immunohistochemistry. In another embodiment, the level of expression of GEP protein is determined by Western Blot analysis.

This invention also provides a method for determining whether a subject is afflicted with Hepatocellular carcinoma (HCC) comprising the steps of (a) determining the level of Granulin-Epithelin Precursor (GEP) protein expression in the tumor cells of the subject; and (b) comparing the level of expression determined in step (a) with the level of expression of GEP protein in normal liver cells of a healthy subject, wherein a higher expression level in step (a) indicates that the subject is afflicted with HCC. In one embodiment, the level of expression of GEP protein is determined by immunohistochemistry. In another embodiment, the level of expression of GEP protein is determined by Western Blot analysis.

This invention also provides a method for determining whether a subject is afflicted with Hepatocellular carcinoma (HCC) comprising the steps of: (a) determining the amount of Granulin-Epithelin Precursor (GEP)-encoding mRNA in the tumor cells of the subject; (b) determining the amount of Granulin-Epithelin Precursor (GEP)-encoding mRNA in the normal liver cells of the subject; and (c) comparing the amount of mRNA determined in step (a) with the amount of mRNA determined in step (b), wherein a greater amount of mRNA in step (a) indicates that the subject is afflicted with HCC. In one embodiment, the amount of mRNA is determined by Quantitative Real-Time Polymerase Chain Reaction using a forward primer, a reverse primer and a probe. The forward primer may comprise, but is not limited to, the nucleic acid sequence 5′-CAA ATG GCC CAC AAC ACT GA-3′ as set forth in SEQ ID NO:2. The reverse primer may comprise, but is not limited to, the nucleic acid sequence 5′-CCC TGA GAC GGT AAA GAT GCA-3′ as set forth in SEQ ID NO:3. The probe may comprise, but is not limited to, the sequence 5′-6FAMCCA CTG CTC TGC CGG CCA CTCMGBNFQ-3′ as set forth in SEQ ID NO:4.

This invention also provides a method for determining whether a subject is afflicted with Hepatocellular carcinoma (HCC) comprising the steps of (a) determining the amount of Granulin-Epithelin Precursor (GEP)-encoding mRNA in the tumor cells of the subject; and (b) comparing the amount of mRNA determined in step (a) with the amount of GEP-encoding mRNA found in normal liver cells of a healthy subject, wherein a greater amount of mRNA in step (a) indicates that the subject is afflicted with HCC. In one embodiment, the amount of mRNA is determined by Quantitative Real-Time Polymerase Chain Reaction using a forward primer, a reverse primer and a probe. The forward primer may comprise, but is not limited to, the nucleic acid sequence 5′-CAA ATG GCC CAC AAC ACT GA-3′ as set forth in SEQ ID NO:2. The reverse primer may comprise, but is not limited to, the nucleic acid sequence 5′-CCC TGA GAC GGT AAA GAT GCA-3′ as set forth in SEQ ID NO:3. The probe may comprise, but is not limited to, the sequence 5′-6FAMCCA CTG CTC TGC CGG CCA CTCMGBNFQ-3′ as set forth in SEQ ID NO:4.

This invention further provides a method for treating a subject afflicted with Hepatocellular carcinoma (HCC) comprising administering to the subject a therapeutically effective amount of an agent which specifically interferes with the expression of the Granulin-Epithelin Precursor (GEP) protein in the tumor cells of the subject. In one embodiment, the agent is a nucleic acid. The nucleic acid may be, but is not limited to, a small interfering RNA, a ribozyme, a DNAzyme or an antisense molecule. The antisense molecule may comprises the nucleic acid sequence GAAGGGGCAG CAACTGGAAG TCCCTGAGAC GGTAAAGATG CAGGAGTGGC CGGCAGAGCA GTGGGCATCA ACCTGGCAGG GGCCACCCAG ATGCCTGCTC AGTGTTGTGG GCCATTTGTC CAGAAGGGGA CGGCAGCAGC TGTAGCTGGC TCCTCCGGGG TCCAGGCAGC AGGCCACAGG GCAGAACTGA CCATCTGGGC ACCGCGTTCC AGCCACCAGC CCTGCTGTTA AGGCCACCCA GCTCACCAGG GTCCACATGG TCTGCCTGCG TCCGACTCCG CGGTCCTTG as set forth in SEQ ID NO:5. In the preferred embodiment, the subject is human.

This invention is illustrated in the Experimental Details section which follows. This section is set forth to aid in an understanding of the invention but is not intended to, and should not be construed to limit in any way the invention as set forth in the claims which follow thereafter.

Experimental Details

Granulin-Epithelin Precursor (GEP) is abundantly and uniquely expressed in hepatocellular carcinoma (HCC), as compared to the surrounding normal liver tissue from HCC patients and normal liver tissue from healthy individuals. Functional studies in two different HCC cell lines (Hep3B and Huh7) demonstrated that GEP down-regulation led to decreased proliferation, tumor invasiveness, and colony forming ability. In vivo experiments using Balb/c athymic mice demonstrated that GEP down-regulation leads to decreased proliferation, and decreased tumorigenicity.

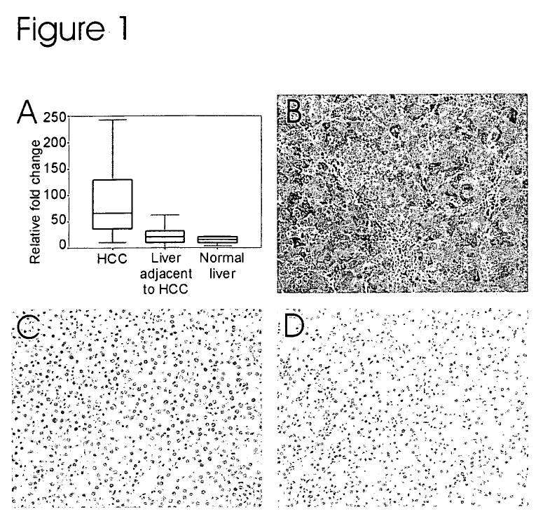

The examination of 110 pairs of HCC and the adjacent normal liver tissues revealed that the RNA levels in the HCC were significantly higher than what in the adjacent normal livers (FIG. 1A). Using entirely normal liver tissues for normalization, it was also shown that the high level of GEP RNA in the tumorigenic tissues was a consequence of over-expression of GEP (FIG. 1A).

High levels of GEP RNA in the HCC tissues positively correlated with the GEP protein expression levels revealed by quantitative real-time PCR and semi-quantitative Western blot scanned by a densitometer (FIG. 1). A majority of HCC tissues showed strong to intermediate expression levels of the GEP protein, while a majority of adjacent liver tissues and entirely normal liver tissues showed weak to zero expression levels of the GEP protein.

TABLE 1
GEP protein expression in human liver samples.

| | HCC patients | Liver | |
|---|---|---|---|
| GEP protein | HCC | adjacent to HCC | Normal |
| expression score | n = 110 | n = 110 | n = 22 |
| 0 (negative signal) | 25 (22.7%) | 72 (65.5%) | 22 (100%) |
| 1 (weak signal) | 17 (15.5%) | 37 (33.6%) | 0 |
| 2 (intermediate signal) | 22 (20.0%) | 1 (0.9%) | 0 |
| 3 (strong signal) | 46 (41.8%) | 0 | 0 |

Abbreviations:
GEP, granulin-epithelin precursor;
HCC, hepatocellular carcinoma.

The over-expression of GEP in HCC was further analyzed according to the clinico-pathological significance. The expression levels of GEP protein were scored by immunohistochemistry, and were classified into the “weak expression (scores≦median)” and “strong expression (score>median)” categories. The inventors demonstrated that strong GEP protein expression was significantly associated with large tumors (>5 cm), venous infiltration and intrahepatic recurrence in the first year (Table 2). In contrast, the GEP expression level was not significantly associated with the serum alpha-fetoprotein (AFP) level, tumor capsule, number of tumor nodules, microsatellite nodules, gender, age of the patients, HBV association (assessed by serum HBsAg), or pTNM stages.

TABLE 2
Clinico-pathological features of HCC in relation to GEP expression.

| | |
|---|---|
| Clinico-pathological | GEP expression |

| parameters | 0-2 | 3 | P |
|---|---|---|---|
| Tumor size | | | |
| Small (≦5 cm) | 30 | 13 | 0.048* |
| Large (>5 cm) | 34 | 33 | |
| Venous infiltration | | | |
| Absent | 37 | 13 | 0.002* |
| Present | 27 | 33 | |
| Intrahepatic recurrence | | | |
| in the first year | | | |
| Yes | 14 | 18 | 0.049* |
| No | 50 | 28 | |
| Serum AFP level | | | |
| Low (≦20 ηg/ml) | 26 | 12 | 0.114 |
| High (>20 ηg/ml) | 38 | 34 | |
| Tumor capsule | | | |
| Absent | 42 | 38 | 0.050 |
| Present | 20 | 7 | |
| Tumor nodules | | | |
| Single | 49 | 35 | 0.954 |
| Multiple | 15 | 11 | |
| Microsatellite nodules | | | |
| Absent | 31 | 20 | 0.502 |
| Present | 31 | 26 | |
| Gender | | | |
| Male | 55 | 34 | 0.113 |
| Female | 9 | 12 | |
| Age | | | |
| Young (≦median, 52) | 34 | 27 | 0.300 |
| Elderly (>median, 52) | 30 | 19 | |
| HBV association | | | |
| Positive for HBsAg | 6 | 4 | 1.000 |
| Negative for HBsAg | 58 | 42 | |
| pTNM stage | | | |
| Early stage (I-II) | 28 | 14 | 0.105 |
| Late stage (III-IV) | 33 | 32 | |

Abbreviations:
HCC, hepatocellular carcinoma;
GEP, granulin-epithelin precursor;
AFP, alpha-fetoprotein;
HBsAg, hepatitis B surface antigen.

Effects of Anti-Sense Oligonucleotide Complementary to GEP

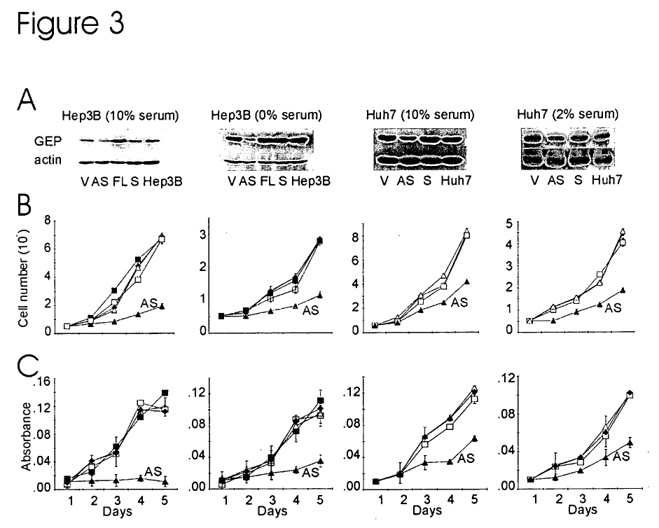

Using the parental Hep3B and Huh7 HCC cell lines as in vitro models, high level of GEP expression was revealed by quantitative RT-PCR. The cell lines were transfected with different constructs for the assessment of GEP expression inhibition. It was demonstrated that GEP expression and protein levels were lowered by the transfected anti-sense fragments (FIG. 3). Moreover, the cell proliferation rate was also examined in serum-containing and serum-limited conditions. The anti-sense transfectants showed a significant decrease in cell proliferation.

TABLE 3
Correlation of GEP level* with cell proliferation^{#} in HCC transfectants.

| | Hep3B | | | | Huh7 | | | |
|---|---|---|---|---|---|---|---|---|
| | 10% serum | | 0% serum | | 10% serum | | | |
| | | Cell | | Cell | | Cell | 2% serum | |
| | | doubling | | doubling | | doubling | | Cell |
| | GEP | (hr) | GEP | (hr) | GEP | (hr) | GEP | doubling (hr) |
| anti- | 0.3 | 40.3 | 0.2 | 62.6 | 0.4 | 42.5 | 0.3 | 49.5 |
| sense | | | | | | | | |
| full- | 2.5 | 22.8 | 1.2 | 39.7 | N.D. | N.D. | N.D. | N.D. |
| length | | | | | | | | |
| sense | 0.9 | 25.3 | 1.1 | 34.3 | 1.1 | 29.1 | 1.0 | 31.3 |
| control | | | | | | | | |
| vector | 0.9 | 23.2 | 1.1 | 40.4 | 1.1 | 32.8 | 1.0 | 31.0 |
| control | | | | | | | | |

GEP, granulin-epithelin precursor;
HCC, hepatocellular carcinoma.
*GEP level of the transfectants referred to the relative fold difference with the parental cells.
^{#}Cell doubling time was assessed during day 3 to 5, since this period was at the log phase of cell proliferation.

The MMT assay by the measurement of mitochondrial activity was used to assess the cellular activity. The anti-sense transfectants demonstrated a marked reduction of cell activity in both the serum-containing and serum-limited conditions, while the full-length transfectants demonstrated a similar cell activity with the respective parental cell lines in both conditions.

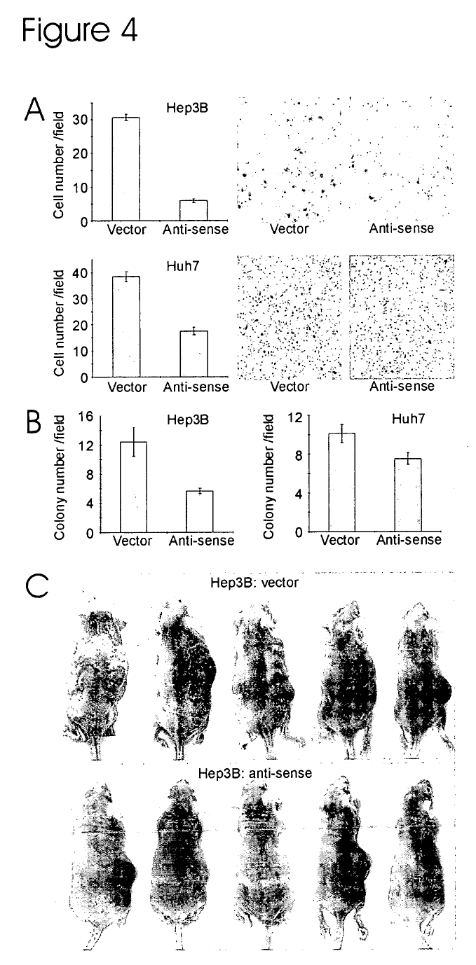

The cell invasion ability was investigated using the Matrigel cell invasion chamber in the two HCC cell lines. A 48 hours incubation period was performed, allowing the 50,000 to migrate and in invade from a serum free medium into the serum containing medium separated by a BD Matrigel Basement Membrane Matrix (BD Biosciences). In Hep3B, the anti-sense transfectants showed a 5.2-fold reduction in cell migration as compared to the empty vector control. Similarly, the anti-sense Huh7 transfectants showed a 2.2-fold reduction in cell migration as compared to the empty vector control (FIG. 4A). These finding clearly demonstrated that the inhibition of GEP expression would result in cell migration reduction and, subsequently, the invasiveness of HCC cells.

The colony-forming ability of the transfectants was assessed in an anchorage-independent condition wherein 50,000 transfected cells were allowed to colonize over 4 weeks. Using a microscope, the number of cells in the colonies formed were counted from at least three independent experiments performed in duplicates. The total cell mass from colonies of Hep3B and Huh7 anti-sense transfectants were significantly reduced by 2.2 and 1.3 folds, respectively, as compared to the empty vector controls (FIG. 4B). This finding clearly demonstrated the functional effects of GEP relating to tumor colony formation.

The tumorigenic potential in the transfectants was assessed in the 4-week old Balb/c athymic mice. Subcutaneous inoculations of 5 millions cells at the dorsal trunk region of the animals were performed. Two measurements of tumor size and body weight were carried out weekly to screen tumor development. The Hep3B anti-sense transfectants developed tumors in 3 out of 5 mice examined, whereas the empty vector transfectants developed tumors with larger size in all 5 mice. All experimental animals were terminated on day 60, and the tumors were surgically removed for net weight determination. The tumor weight of the antisense group was significantly reduced by 7.7-fold as compared to the vector control group.

These studies demonstrate that GEP positively regulates the cell proliferation rate, cell activity, cell invasion, colony formation, as well as tumorigenic potential. The functional data further corroborate the clinical observations that strong GEP expression is always associated with large HCC size and the presence of venous infiltration. It was therefore demonstrated that GEP plays a major role in hepato-carcinogenesis, contributing to different tumor stages from proliferation to the subsequent invasion and metastasis.

Patients and Sample Collections

Tissue samples from liver tumors, non-tumor liver tissues adjacent to tumors, cirrhotic liver from non-cancer patients and normal livers were obtained during operation. Distribution of the pTNM stages and other clinico-pathological parameters are listed in Table 1. Normal liver specimens were collected in transplant operations. The organ donors had no underlying liver diseases and were negative for hepatitis B serology. Each tissue specimen, 0.5-1 cm³, was divided into 3 equal portions. One portion was formalin-fixed and paraffin-embedded for histological and immunohistochemical studies. Two portions were snap-frozen in liquid nitrogen and stored at −70° C. until use.

RNA Extraction from Tumor Samples

Total RNA was extracted using TRIZOL reagent (Invitrogen, USA) according to the manufacturer's protocol. Briefly, frozen tissue sample was put into 10 ml TRIZOL reagent and homogenized immediately. The homogenized sample was passed through a syringe to shear the genomic DNA and allowed to stand in room temperature for 5 minutes. Then the homogenate was centrifuged at 400 rpm for 30 minutes at 4° C. The cleared homogenate solution was transferred to a clean tube and 2 ml chloroform was added. After throughout mixing, the mixture was centrifuged at 4500 rpm for 30 minutes at 4° C. to separate the RNA-containing aqueous phase. The aqueous phase was transferred to a clean tube and 5 ml isopropanol was added to precipitate RNA from the sample. The precipitated RNA was collected by centrifugation and washed twice with 75% ethanol. At the end of the procedure, RNA was dissolved in DEPC-water for subsequent experiments.

First Strand cDNA Synthesis

The first strand cDNA was synthesized from 0.5 μg of total RNA from the samples using High Capacity cDNA Achive kit (Applied Biosystems, USA) according to the manufacturer's instruction. Total RNA samples were first treated with 1 unit DNase I at room temperature for 15 minutes. Then the reaction was stopped by adding EDTA solution and heating at 70° C. for 10 minutes. The DNase I treated total RNA samples were added to a reverse-transcription reaction mix containing 1×RT buffer, 4 mM dNTP mix, 1× random primer, 125 units of MultiScribe RT. The mixture was incubated at 25° C. for 10 minutes and 37° C. for 2 hours to synthesize the first strand cDNA.

Immunohistochemical Staining

Immunohistochemistry was performed using the Dako Envision Plus System (Dako, Carpinteria, Calif.) following the manufacturer's instruction with modifications. Briefly, antigen retrieval was performed by microwave with sections immersed in citrate buffer. Followed by endogenous peroxidase blocking, primary antibody was applied. The signal was detected by horseradish peroxidase-conjugated secondary antibody and color was developed using diaminobenzidine as the chromogen. The tissue sections were then counterstained with hematoxylin. For GEP, 2 ug/ml of polyclonal antibody GEP (AGI, Sunnyvale, Calif.) was used. For alpha-fetoprotein (AFP), polyclonal antibody (Dako) in 1:50 dilution was used. For p53 detection, monoclonal antibody DO-7 (Dako) in 1:50 dilution was used.

Western Blot Analysis

Total protein of 30 μg was separated in 10% SDS-PAGE gel and transferred to polyvinylidene difluoride membrane (Millipore, Bedford, Mass.). The blots were blocked with 10% non-fat dry milk, probed against polyclonal GEP antibody, followed by anti-rabbit IgG conjugated with horseradish peroxidase (Sigma-Aldrich, St. Louis, Mo.). The bands were visualized using the Enhanced Chemiluminescence Western Blotting Detection Kit (Amersham Biosciences, Buckinghamshire, UK) according to the manufacturer's instructions and exposed on the Hyperfilm™ (Amersham Biosciences). The relative levels of protein were quantified by densitometric scanning of the exposed films, using a gel-imaging system and the UVP GelWorks ID Intermediate version 3.01 (Ultra Violet Products Ltd., Cambridge, UK).

Quantitative Real-Time PCR

Real-time quantitative multiplex RT-PCR was performed using an ABI PRISM 7700 Sequence Detection System (Applied Biosystems, USA). Five microliters of 1:60 fold diluted first strand cDNA was used in the assay of GEP gene expression. Primers and probe for 18s rRNA from Pre-Developed TaqMan Assay Reagents was used as endogenous control of all samples in all the PCRs. In each 25 μl PCR reaction, it contains 1×PCR buffer 11, 5.5 mM MgCl₂, 0.2 mM dATP, dCTP, dGTP, 0.4 mM dUTP, 0.625 unit of AmpliTaq Gold. The optimal primers and probe concentrations of target genes and optimal 18s rRNA control dilution for the gene expression assay were as follows:

```
SEQ ID NO:1
      1 GTAGTCTGAG CGCTACCCGG TTGCTGCTGC CCAAGGACCG CGGAGTCGGA CGCAGGCAGA
    61 CCATGTGGAC CCTGGTGAGC TGGGTGGCCT TAACAGCAGG GCTGGTGGCT GGAACGCGGT
  121 GCCCAGATGG TCAGTTCTGC CCTGTGGCCT GCTGCCTGGA CCCCGGAGGA GCCAGCTACA
  181 GCTGCTGCCG TCCCCTTCTG GACAAATGGC CCACAACACT GAGCAGGCAT CTGGGTGGCC
  241 CCTGCCAGGT TGATGCCCAC TGCTCTGCCG GCCACTCCTG CATCTTTACC GTCTCAGGGA
  301 CTTCCAGTTG CTGCCCCTTC CCAGAGGCCG TGGCATGCGG GGATGGCCAT CACTGCTGCC
  361 CACGGGGCTT CCACTGCAGT GCAGACGGGC GATCCTGCTT CCAAAGATCA GGTAACAACT
  421 CCGTGGGTGC CATCCAGTGC CCTGATAGTC AGTTCGAATG CCCGGACTTC TCCACGTGCT
  481 GTGTTATGGT CGATGGCTCC TGGGGGTGCT GCCCCATGCC CCAGGCTTCC TGCTGTGAAG
  541 ACAGGGTGCA CTGCTGTCCG CACGGTGCCT TCTGCGACCT GGTTCACACC CGCTGCATCA
  601 CACCCACGGG CACCCACCCC CTGGCAAAGA AGCTCCCTGC CCAGAGGACT AACAGGGCAG
  661 TGGCCTTGTC CAGCTCGGTC ATGTGTCCGG ACGCACGGTC CCGGTGCCCT GATGGTTCTA
  721 CCTGCTGTGA GCTGCCCAGT GGGAAGTATG GCTGCTGCCC AATGCCCAAC GCCACCTGCT
  781 GCTCCGATCA CCTGCACTGC TGCCCCCAAG ACACTGTGTG TGACCTGATC CAGAGTAAGT
  841 GCCTCTCCAA GGAGAACGCT ACCACGGACC TCCTCACTAA GCTGCCTGCG CACACAGTGG
  901 GGGATGTGAA ATGTGACATG GAGGTGAGCT GCCCAGATGG CTATACCTGC TGCCGTCTAC
  961 AGTCGGGGGC CTGGGGCTGC TGCCCTTTTA CCCAGGCTGT GTGCTGTGAG GACCACATAC
1021 ACTGCTGTCC CGCGGGGTTT ACGTGTGACA CGCAGAAGGG TACCTGTGAA CAGGGGCCCC
1081 ACCAGGTGCC CTGGATGGAG AAGGCCCCAG CTCACCTCAG CCTGCCAGAC CCACAAGCCT
1141 TGAAGAGAGA TGTCCCCTGT GATAATGTCA GCAGCTGTCC CTCCTCCGAT ACCTGCTGCC
1201 AACTCACGTC TGGGGAGTGG GGCTGCTGTC CAATCCCAGA GGCTGTCTGC TGCTCGGACC
1261 ACCAGCACTG CTGCCCCCAG GGCTACACGT GTGTAGCTGA GGGGCAGTGT CAGCGAGGAA
1321 GCGAGATCGT GGCTGGACTG GAGAAGATGC CTGCCCGCCG GGCTTCCTTA TCCCACCCCA
1381 GAGACATCGG CTGTGACCAG CACACCAGCT GCCCGGTGGG GCAGACCTGC TGCCCGAGCC
1441 TGGGTGGGAG CTGGGCCTGC TGCCAGTTGC CCCATGCTGT GTGCTGCGAG GATCGCCAGC
1501 ACTGCTGCCC GGCTGGCTAC ACCTGCAACG TGAAGGCTCG ATCCTGCGAG AAGGAAGTGG
1561 TCTCTGCCCA GCCTGCCACC TTCCTGGCCC GTAGCCCTCA CGTGGGTGTG AAGGACGTGG
1621 AGTGTGGGGA AGGACACTTC TGCCATGATA ACCAGACCTG CTGCCGAGAC AACCGACAGG
1681 GCTGGGCCTG CTGTCCCTAC CGCCAGGGCG TCTGTTGTGC TGATCGGCGC CACTGCTGTC
1741 CTGCTGGCTT CCGCTGCGCA GCCAGGGGTA CCAAGTGTTT GCGCAGGGAG GCCCCGCGCT
1801 GGGACGCCCC TTTGAGGGAC CCAGCCTTGA GACAGCTGCT GTGAGGGACA GTACTGAAGA
1861 CTCTGCAGCC CTCGGGACCC CACTCGGAGG GTGCCCTCTG CTCAGGCCTC CCTAGCACCT
1921 CCCCCTAACC AAATTCTCCC TGGACCCCAT TCTGAGCTCC CCATCACCAT GGGAGGTGGG
1981 GCCTCAATCT AAGGCCTTCC CTGTCAGAAG GGGGTTGTGG CAAAAGCCAC ATTACAAGCT
2041 GCCATCCCCT CCCCGTTTCA GTGGACCCTG TGGCCAGGTG CTTTTCCCTA TCCACAGGGG
2101 TGTTTGTGTG TGTGCGCGTG TGCGTTTCAA TAAAGTTTGT ACACTTTC
NOTE FOR SEQ ID NO:1
POLYMORPHISM AT 446: T OR C
POLYMORPHISM AT 1922: T OR C
SEQ ID NO:2
5′-CAA ATG GCC GAC AAC ACT GA-3′(0.2 μM)
SEQ ID NO:3
5′-CCC TGA GAC GGT AAA GAT GCA-3′(0.2 μM)
SEQ ID NO:4
5′-6FAMCCA CTG CTC TGC CGG CCA CTCMGBNFQ-3′
(0.2 μM)
```

18s control: 1×

The conditions for the quantitative real-time PCR were as follows: 95° C. for 10 minutes and 40 cycles of 95° C. for 15 seconds and 60° C. for 1 minute. The amplification plots of the PCR reaction generated by the software [Applied Biosystems] were used to determine the threshold cycle (CT). The CT value represented the PCR cycles at which an increase in reporter fluorescence above a baseline signal can first be detected.

Cell Culture and Transfection of Anti-Sense GEP cDNA

The full-length GEP cDNA (SEQ ID NO:1) cloned in pCMV6-XL5 (OriGene Technologies Inc., Rockville, Md.) was used as the template for assembly of different GEP constructs into pcDNA3.1(+) (Invitrogen, Carlsbad, Calif.). The full-length GEP was subcloned by using the Not1 and XbaI restriction sites. The N-terminal fragment (size of 290 bp, corresponding to position −31 to 258 bp)^{7,8} was generated by polymerase chain reaction (PCR), and sub-cloned in antisense and sense orientation to generate the respective constructs. Two human HCC cell lines, Hep3B (American Tissue Culture Collection, Rockville, Md.) and Huh7 (Health Science Research Resources Bank, Osaka, Japan), were used. Hep3B is p53-deficient, whereas Huh7 contains the mutant p53 with over-expression of the p53 protein. These two cell lines were used to test if the GEP function was p53-dependent. The cells were maintained under standard culture condition with serum-containing DMEM (supplemented with 10% FBS, 50 U/ml Penicillin G and 50 μg/ml Streptomycin). The cells were transfected with LipofectAMINE (Invitrogen) according to the manufacturer's instruction: 1, anti-sense fragment to decrease the GEP level; 2, full-length for over-expression of GEP; 3, sense fragment as control for anti-sense experiment; 4, empty vector as control for all the transfection experiments. Stable clones were selected by G418. The GEP protein level and proliferation were assessed in serum-containing (10%), serum-limited (0% serum for Hep3B, and 2% serum for Huh7 as cell proliferation in 0% serum was insignificant) conditions.

SEQ ID NO:5
GAAGGGGCAG CAACTGGAAG TCCCTGAGAC GGTAAAGATG CAGGAGTGGC CGGCAGAGCA
GTGGGCATCA ACCTGGCAGG GGCCACCCAG ATGCCTGCTC AGTGTTGTGG GCCATTTGTC
CAGAAGGGGA CGGCAGCAGC TGTAGCTGGC TCCTCCGGGG TCCAGGCAGC AGGCCACAGG
GCAGAACTGA CCATCTGGGC ACCGCGTTCC AGCCACCAGC CCTGCTGTTA AGGCCACCCA
GCTCACCAGG GTCCACATGG TCTGCCTGCG TCCGACTCCG CGGTCCTTG

In Vitro Functional Analysis of GEP-Transfected HCC Cells

Cell proliferation was assayed by seeding fifty thousand cells into 6-well plates. Cells were harvested every day for 5 consecutive days, and viable cells were counted by trypan blue exclusion. Cell activity was measured via mitochondrial dehydrogenase activity performed by MTT assay,^{18,19} in which five thousand cells were seeded into 96-well plates and assayed for 5 consecutive days. The cell invasion ability was determined using the BioCoat Matrigel invasion chamber (BD Biosciences, Bedford, Mass.), in which the chamber membrane filter (8 μm pore size) was coated with the BD Matrigel™ Basement Membrane Matrix (BD Biosciences). The upper chamber was loaded with fifty thousand cells in 2 ml serum-free medium, whereas the lower chamber was filled with 2 ml serum-containing medium. After 48 hours of standard incubation, non-invading cells on the upper surface of the membrane were removed with cotton swabs. Invading cells on the lower surface of the membrane were washed in PBS, fixed in Carnoy's solution, and stained with hemotoxylin and eosin. The invading cells were counted under the microscope in 10 randomly selected fields for each membrane filter (×100 magnification). Anchorage-independent growth was assessed by colony formation ability in soft agar.²⁰ The agar base of 1.5 ml in a 6-well plate was formed by mixing an equal volume of 1.6% low-melting agar (USB) and 2×DMEM supplemented with 20% FBS. The five thousand cells were suspended in 1.5 ml soft agar (mixture containing 2×DMEM supplemented with 20% FBS, and 0.8% low-melting agar) and overlaid on the agar base. After 4 weeks, colonies over 15 cells were counted under the microscope in 10 fields per well. Each data point for in vitro experiments represented results from at least three independent experiments performed in duplicates.

In Vivo Functional Analysis of GEP-Transfected HCC Cells

Balb/c athymic nude mice of 4 weeks old were used to test the in vivo tumorigenicity potential of the transfectants.²¹ The study protocol was approved by the Committee on the Use of Live Animals for Teaching and Research at the University of Hong Kong. Five million cells were inoculated subcutaneously at the dorsal region of the trunk of each animal. The tumor size and body weight were measured twice weekly. The mice were terminated on day 60 with the tumor harvested for further examination. Each of the experimental group contained 5 mice.

### REFERENCES

1. Pisani P, Parkin D M, Bray F, Ferlay J. Estimates of the worldwide mortality from 25 cancers in 1990. Int J Cancer 1999; 83:18-29.
2. Staib F, Hussain S P, Hofseth L J, Wang X W, Harris C C. TP53 and liver carcinogenesis. Hum Mutat 2003; 21:201-216.
3. Thorgeirsson S S, Grisham J W. Molecular pathogenesis of human hepatocellular carcinoma. Nat Genet 2002; 31:339-346.
4. Buendia M A. Genetics of hepatocellular carcinoma. Semin Cancer Biol 2000; 10:185-200.
5. Chen X, Cheung S T, So S, Fan S T, Barry C, Higgins J, Lai K M, Ji J, Dudoit S, Ng 10, Van De Rijn M, Botstein D, Brown P O. Gene expression patterns in human liver cancers. Mol Biol Cell 2002; 13:1929-1939.
6. Zhou J, Gao G, Crabb J W, Serrero G. Purification of an autocrine growth factor homologous with mouse epithelin precursor from a highly tumorigenic cell line. J Biol Chem 1993; 268:10863-10869.
7. Zhang H, Serrero G. Inhibition of tumorigenicity of the teratoma PC cell line by transfection with antisense cDNA for PC cell-derived growth factor (PCDGF, epithelin/granulin precursor). Proc NatI Acad Sci USA 1998; 95:14202-14207.
8. Lu R, Serrero G. Inhibition of PC cell-derived growth factor (PCDGF, epithelin/granulin precursor) expression by antisense PCDGF cDNA transfection inhibits tumorigenicity of the human breast carcinoma cell line MDA-MB-468. Proc Natl Acad Sci USA 2000; 97:3993-3998.
9. Chen Y J, Yeh S H, Chen J T, Wu C C, Hsu M T, Tsai S F, Chen P J, Lin C H. Chromosomal changes and clonality relationship between primary and recurrent hepatocellular carcinoma. Gastroenterology 2000; 119:431-440.
10. Kato A, Kubo K, Kurokawa F, Okita K, Oga A, Murakami T. Numerical aberrations of chromosomes 16, 17, and 18 in hepatocellular carcinoma: a FISH and FCM analysis of 20 cases. Dig Dis Sci 1998; 43:1-7.
11. Sobin L H, Whitekind C. TNM classification of malignant tumours. 6th ed. New York: John Wiley, 2002.
12. Bustin S A. Absolute quantification of mRNA using real-time reverse transcription polymerase chain reaction assays. J Mol Endocrinol 2000; 25:169-193.
13. Goidin D, Mamessier A, Staquet M J, Schmitt D, Berthier-Vergnes O. Ribosomal 18S RNA prevails over glyceraldehyde-3-phosphate dehydrogenase and beta-actin genes as internal standard for quantitative comparison of mRNA levels in invasive and noninvasive human melanoma cell subpopulations. Anal Biochem 2001; 295:17-21.
14. Livak K J, Schmittgen T D. Analysis of relative gene expression data using real-time quantitative PCR and the 2(-Delta Delta C(T)) Method. Methods 2001; 25:402-408.
15. Ng 10, Chung L P, Tsang S W, Lam C L, Lai E C, Fan S T, Ng M. p53 gene mutation spectrum in hepatocellular carcinomas in Hong Kong Chinese. Oncogene 1994; 9:985-990.
16. Hollstein M, Sidransky D, Vogelstein B, Harris C C. p53 mutations in human cancers. Science 1991; 253:49-53.
17. Lehman T A, Bennett W P, Metcalf R A, Welsh J A, Ecker J, Modali R V, Ullrich S, Romano J W, Appella E, Testa J R, Gerwin B I, Harris C C. p53 mutations, ras mutations, and p53-heat shock 70 protein complexes in human lung carcinoma cell lines. Cancer Res 1991; 51:4090-4096.
18. Bank U, Reinhold D, Ansorge S. Measurement of cellular activity with the MTT test. Optimization of the method. Allerg Immunol (Leipz) 1991; 37:119-123.
19. Loveland B E, Johns T G, Mackay I R, Vaillant F, Wang Z X, Hertzog P J. Validation of the MTT dye assay for enumeration of cells in proliferative and antiproliferative assays. Biochem Int 1992; 27:501-510.
20. Sapi E, Flick M B, Rodov S, Gilmore-Hebert M, Kelley M, Rockwell S, Kacinski B M. Independent regulation of invasion and anchorage-independent growth by different autophosphorylation sites of the macrophage colony-stimulating factor 1 receptor. Cancer Res 1996; 56:5704-5712.
21. Cheung S T, Huang D P, Hui A B, Lo K W, Ko C W, Tsang Y S, Wong N, Whitney B M, Lee J C. Nasopharyngeal carcinoma cell line (C666-1) consistently harbouring Epstein-Barr virus. Int J Cancer 1999; 83:121-126.
22. Hirohashi S, Bulm H E, Ishak K G, Deugnier Y, Kojiro M, Laurent Pulg P, Wanless I R, Fischer H P, Theise N D, Sakamoto M, Tsukuma H. Hepatocellular carcinoma. In: Hamilton S R and Aaltonen L A, ed. World Health Organization Classification of Tumours. Pathology and Genetics of Tumours of the Digestive System. Geneva, Switzerland: World Health Organization, 2000:158-172.
23. Harada T, Shigemura T, Kodama S, Higuchi T, Ikeda S, Okazaki M. Hepatic resection is not enough for hepatocellular carcinoma. A follow-up study of 92 patients. J Clin Gastroenterol 1992; 14:245-250.
24. Poon R T, Fan S T, Lo C M, Liu C L, Wong J. Intrahepatic recurrence after curative resection of hepatocellular carcinoma: long-term results of treatment and prognostic factors. Ann Surg 1999; 229:216-222.
25. Ng 10, Lai E C, Fan S T, Ng M M, So M K. Prognostic significance of pathologic features of hepatocellular carcinoma. A multivariate analysis of 278 patients. Cancer 1995; 76:2443-2448.
26. Tsai T J, Chau G Y, Lui W Y, Tsay S H, King K L, Loong C C, Hsia C Y, Wu C W. Clinical significance of microscopic tumor venous invasion in patients with resectable hepatocellular carcinoma. Surgery 2000; 127:603-608.
27. Poon R T, Fan S T, Ng 10, Lo C M, Liu C L, Wong J. Different risk factors and prognosis for early and late intrahepatic recurrence after resection of hepatocellular carcinoma. Cancer 2000; 89:500-507.
28. Poon R T, Ng 10, Fan S T, Lai E C, Lo C M, Liu C L, Wong J. Clinicopathologic features of long-term survivors and disease-free survivors after resection of hepatocellular carcinoma: a study of a prospective cohort. J Clin Oncol 2001; 19:3037-3044.
29. Jones M B, Michener C M, Blanchette J O, Kuznetsov V A, Raffeld M, Serrero G, Emmert-Buck M R, Petricoin E F, Krizman D B, Liotta L A, Kohn E C. The granulin-epithelin precursor/PC-cell-derived growth factor is a growth factor for epithelial ovarian cancer. Clin Cancer Res 2003; 9:44-51.
30. Donald C D, Laddu A, Chandham P, Lim S D, Cohen C, Amin M, Gerton G L, Marshall F F, Petros J A. Expression of progranulin and the epithelin/granulin precursor acrogranin correlates with neoplastic state in renal epithelium. Anticancer Res 2001; 21:3739-3742.
31. He Z, Bateman A. Progranulin gene expression regulates epithelial cell growth and promotes tumor growth in vivo. Cancer Res 1999; 59:3222-3229.
32. He Z, Ismail A, Kriazhev L, Sadvakassova G, Bateman A. Progranulin (PC-cell-derived growth factor/acrogranin) regulates invasion and cell survival. Cancer Res 2002; 62:5590-5596.
33. He Z, Ong C H, Halper J, Bateman A. Progranulin is a mediator of the wound response. Nat Med 2003; 9:225-229.
34. Daniel R, Daniels E, He Z, Bateman A. Progranulin (acrogranin/PC cell-derived growth factor/granulin-epithelin precursor) is expressed in the placenta, epidermis, microvasculature, and brain during murine development. Dev Dyn 2003; 227:593-599.
35. Bourdon J C, D'Errico A, Paterlini P, Grigioni W, May E, Debuire B. p53 protein accumulation in European hepatocellular carcinoma is not always dependent on p53 gene mutation. Gastroenterology 1995; 108:1176-1182.
36. Serrero G. Autocrine growth factor revisited: PC-cell-derived growth factor (progranulin), a critical player in breast cancer tumorigenesis. Biochem Biophys Res Commun 2003; 308:409-413.
37. Lu R, Serrero G. Mediation of estrogen mitogenic effect in human breast cancer MCF-7 cells by PC-cell-derived growth factor (PCDGF/granulin precursor). Proc Natl Acad Sci USA 2001; 98:142-147.
38. Ong C H, Bateman A. Progranulin (Granulin-epithelin precursor, PC-cell derived growth factor, Acrogranin) in proliferation and tumorigenesis. Histol Histopathol 2003; 18:1275-1288.
39. Zanocco-Marani T, Bateman A, Romano G, Valentinis B, He Z H, Baserga R. Biological activities and signaling pathways of the granulin/epithelin precursor. Cancer Res 1999; 59:5331-5340.
40. Fang L, Li G, Liu G, Lee S W, Aaronson S A. p53 induction of heparin-binding EGF-like growth factor counteracts p53 growth suppression through activation of MAPK and PI3K/Akt signaling cascades. EMBO J 2001; 20:1931-1939.
41. Huynh H T, Nguyen T T, Chow P K, Tan P H, Soo K C, Tran E. Over-expression of MEK-MAPK in hepatocellular carcinoma: Its role in tumor progression and apoptosis. BMC Gastroenterol 2003; 3:19.

1. A method for determining whether an agent causes a reduction in the expression of Granulin-Epithelin Precursor (GEP) protein in a cell, comprising the steps of:
(a) contacting the cell with the agent under conditions which, in the absence of the agent, permit expression of the GEP protein;
(b) after a suitable period of time, determining the amount of expression in the cell of the GEP protein; and
(c) comparing the amount of expression determined in step (b) with the amount of expression which occurs in the absence of the agent, whereby a reduced amount of expression in the presence of the agent indicates that the agent causes a reduction in the expression of the GEP protein.

2. The method of claim 1, wherein the cell is present in a cell culture.

3. The method of claim 1, wherein the cell is a tumor cell.

4. The method of claim 1, wherein determining the amount of expression is performed by determining the amount of GEP protein-encoding mRNA in the cell.

5. The method of claim 1, wherein determining the amount of expression is performed by determining the amount of GEP protein in the cell.

6. The method of claim 5, wherein determining the amount of GEP protein in the cell is performed using an antibody specific for the GEP protein.

7. A method for determining whether an agent causes a reduction in the activity of a Granulin-Epithelin Precursor (GEP) protein in a cell, comprising the steps of:
(a) contacting the cell with the agent under conditions which, in the absence of the agent, permit activity of the GEP protein;
(b) determining the amount of activity of the GEP protein in the cell; and
(c) comparing the amount of activity determined in step (b) with the amount of activity which occurs in the absence of the agent, whereby a reduced amount of activity in the presence of the agent indicates that the agent causes a reduction in the activity of the GEP protein.

8. The method of claim 7, wherein the cell is present in a cell culture.

9. The method of claim 7, wherein the cell is a tumor cell.

10. A method for reducing the expression of Granulin-Epithelin Precursor (GEP) protein in a cell comprising introducing into the cell an agent which specifically interferes with the expression of the GEP protein in the cell.

11. The method of claim 10, wherein the cell is present in a cell culture.

12. The method of claim 10, wherein the cell is a tumor cell.

13. The method of claim 10, wherein the agent is a nucleic acid.

14. The method of claim 13, wherein the nucleic acid is a small interfering RNA, a ribozyme, DNAzyme or an antisense molecule.

15. The method of claim 14, wherein the antisense molecule comprises the nucleic acid sequence as set forth in SEQ ID NO:5.

16. A method for determining whether a subject is afflicted with Hepatocellular carcinoma (HCC) comprising the steps of:
(a) determining the level of Granulin-Epithelin Precursor (GEP) protein expression in the tumor cells of the subject;
(b) determining the level of Granulin-Epithelin Precursor (GEP) protein expression in the normal liver cells of the subject; and
(c) comparing the level of expression determined in step (a) with the level of expression determined in step (b), wherein a higher expression level in step (a) indicates that the subject is afflicted with HCC.

17. The method of claim 16, wherein the level of expression of GEP protein is determined by immunohistochemistry or Western Blot analysis.

18. A method for determining whether a subject is afflicted with Hepatocellular carcinoma (HCC) comprising the steps of:
(a) determining the level of Granulin-Epithelin Precursor (GEP) protein expression in the tumor cells of the subject; and
(b) comparing the level of expression determined in step (a) with the level of expression of GEP protein in normal liver cells of a healthy subject, wherein a higher expression level in step (a) indicates that the subject is afflicted with HCC.

19. The method of claim 18, wherein the level of expression of GEP protein is determined by immunohistochemistry or Western Blot analysis.

20. A method for determining whether a subject is afflicted with Hepatocellular carcinoma (HCC) comprising the steps of:
(a) determining the amount of Granulin-Epithelin Precursor (GEP)-encoding mRNA in the tumor cells of the subject;
(b) determining the amount of Granulin-Epithelin Precursor (GEP)-encoding mRNA in the normal liver cells of the subject; and
(c) comparing the amount of mRNA determined in step (a) with the amount of mRNA determined in step (b), wherein a greater amount of mRNA in step (a) indicates that the subject is afflicted with HCC.

21. The method of claim 20, wherein the amount of mRNA is determined by Quantitative Real-Time Polymerase Chain Reaction using a forward primer, a reverse primer and a probe.

22. The method of claim 21, wherein the forward primer comprises the nucleic acid sequence as set forth in SEQ ID NO:2.

23. The method of claim 21, wherein the reverse primer comprises the nucleic acid sequence as set forth in SEQ ID NO:3.

24. The method of claim 21, wherein the probe comprises the nucleic acid sequence as set forth in SEQ ID NO:4.

25. A method for determining whether a subject is afflicted with Hepatocellular carcinoma (HCC) comprising the steps of:
(a) determining the amount of Granulin-Epithelin Precursor (GEP)-encoding mRNA in the tumor cells of the subject; and
(b) comparing the amount of mRNA determined in step (a) with the amount of GEP-encoding mRNA found in normal liver cells of a healthy subject, wherein a greater amount of mRNA in step (a) indicates that the subject is afflicted with HCC.

26. The method of claim 25, wherein the amount of mRNA is determined by Quantitative Real-Time Polymerase Chain Reaction using a forward primer, a reverse primer and a probe.

27. The method of claim 26, wherein the forward primer comprises the nucleic acid sequence as set forth in SEQ ID NO:2.

28. The method of claim 26, wherein the reverse primer comprises the nucleic acid sequence as set forth in SEQ ID NO:3.

29. The method of claim 26, wherein the probe comprises the nucleic acid sequence as set forth in SEQ ID NO:4.

30. A method for treating a subject afflicted with Hepatocellular carcinoma (HCC) comprising administering to the subject a therapeutically effective amount of an agent which specifically interferes with the expression of the Granulin-Epithelin Precursor (GEP) protein in the tumor cells of the subject.

31. The method of claim 30, wherein the agent is a nucleic acid.

32. The method of claim 31, wherein the nucleic acid is a small interfering RNA, a ribozyme, DNAzyme or an antisense molecule.

33. The method of claim 32, wherein the antisense molecule comprises the nucleic acid sequence as set forth in SEQ ID NO:5.

34. The method of claim 30, wherein the subject is human.


SEQUENCE LISTING (5 sequences)

SEQ ID NO: 1 - DNA, 2148 bp, Human
gtagtctgagcgctacccggttgctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttc

SEQ ID NO: 2 - DNA, 20 bp, Artificial
  PCR Primer
caaatggcccacaacactga

SEQ ID NO: 3 - DNA, 21 bp, Artificial
  PCR Primer
ccctgagacggtaaagatgca

SEQ ID NO: 4 - DNA, 21 bp, Artificial
  GEP Probe
ccactgctctgccggccactc

SEQ ID NO: 5 - DNA, 289 bp, Artificial
  Antisense sequence
gaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtctgcctgcgtccgactccgcggtccttg